SEQUENCE LISTING (16 sequences)

SEQ ID NO: 1 - protein, 708 aa, SV40 virus
MDKVLNREESLQLMDLLGLERSAWGNIPLMRKAYLKKCKEFHPDKGGDEEKMKKMNTLYKKMEDGVKYAHQPDFGGFWDATEIPTYGTDEWEQWWNAFNEENLFCSEEMPSSDDEATADSQHSTPPKKKRKVEDPKDFPSELLSFLSHAVFSNRTLACFAIYTTKEKAALLYKKIMEKYSVTFISRHNSYNHNILFFLTPHRHRVSAINNYAQKLCTFSFLICKGVNKEYLMYSALTRDPFSVIEESLPGGLKEHDFNPEEAEETKQVSWKLVTEYAMETKCDDVLLLLGMYLEFQYSFEMCLKCIKKEQPSHYKYHEKYYANAAIFADSKNQKTICQQAVDTVLAKKRVDSLQLTREQMLTNRFNDLLDRMDIMFGSTGSADIEEWMAGVAWLHCLLPKMDSVVYDFLKCMVYNIPKKRYWLFKGPIDSGKTTLAAALLELCGGKALNVNLPLDRLNFELGVAIDQFLVVFEDVKGTGGESRDLPSGQGINNLDNLRDYLDGSVKVNLEKKHLNKRTQIFPPGIVTMNEYSVPKTLQARFVKQIDFRPKDYLKHCLERSEFLLEKRIIQSGIALLLMLIWYRPVAEFAQSIQSRIVEWKERLDKEFSLSVYQKMKFNVAMGIGVLDWLRNSDDDDEDSQENADKNEDGGEKNMEDSGHETGIDSQSQGSFQAPQSSQSVHDHNQPYHICRGFTCFKKPPTPPPEPET

SEQ ID NO: 2 - DNA, 64 bp, Artificial Sequence
  Synthetic Oligonucleotide
gatccccgatgcatttgaagcccagtttcaagagaactgggcttcaaatgcatctttttggaaa

SEQ ID NO: 3 - DNA, 64 bp, Artificial Sequence
  Synthetic Oligonucleotide
agcttttccaaaaagatgcatttgaagcccagttctcttgaaactgggcttcaaatgcatcggg

SEQ ID NO: 4 - DNA, 23 bp, Artificial Sequence
  Synthetic Oligonucleotide
gaugcauuugaagcccagudtdt

SEQ ID NO: 5 - DNA, 23 bp, Artificial Sequence
  Synthetic Oligonucleotide
acugggcuucaaaugcaucdtdt

SEQ ID NO: 6 - protein, 5 aa, SV40 Virus
ATEIP

SEQ ID NO: 7 - protein, 16 aa, SV40 Virus
TYGTDEWEQWWNAFNE

SEQ ID NO: 8 - protein, 9 aa, SV40 Virus
NLFCSEEMP

SEQ ID NO: 9 - protein, 20 aa, BK Virus
TYGTEEWESWWSSFNEKWDE

SEQ ID NO: 10 - protein, 8 aa, BK Virus
DLFCHEDM

SEQ ID NO: 11 - protein, 20 aa, JC Virus
TYGTDEWESWWNTFNEKWDE

SEQ ID NO: 12 - protein, 7 aa, JC Virus
DLFCHEE

SEQ ID NO: 13 - protein, 26 aa, BP Virus
GTSQWEQWWEEFNQGFDEQDLHCDEE

SEQ ID NO: 14 - protein, 18 aa, Kilham Virus
SYGTPEWDEWWKEFNKDF

SEQ ID NO: 15 - protein, 9 aa, Kilham Virus
DLFCNEAFD

SEQ ID NO: 16 - protein, 25 aa, Py Virus
DDYWSFSYGSKYFTREWNDFFRKWD

(19) United States
(12) Patent Application Publication
(10) Pub. No.: US 2009/0252715 A1
(43) Pub. Date: Oct. 8, 2009

(54) IMMORTALIZATION OF MAMMALIAN CELLS

(76) Inventors: THOMAS ROBERTS, Boston, MA (US); Ole Gjoerup, Boston, MA (US); Parmjit Jat, New York, NY (US); Marina Cotsiki, Athens (GR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/482,244

(22) Filed: Jun. 10, 2009

Related U.S. Application Data
(62) Division of application No. 10/570,126, filed on Jun. 12, 2007, now abandoned.
(60) Provisional application No. 60/502,514, filed on Sep. 12, 2003.

(51) Int. Cl. A61K 48/00; C07K 14/00; C07H 21/04; C12N 5/10

(52) U.S. Cl. 424/93.21; 530/350; 536/23.1; 435/325; 435/366; 536/23.2

(57) ABSTRACT

Genomic instability in T antigen expressing cells can be overcome by modifying the gene expressing T antigen so that it lacks Bub1 binding. Stable cell lines can be produced by incorporation of the modified T antigen gene, preferably together with the catalytic sub-unit of the telomerase construct.

### CROSS-REFERENCE TO RELATED APPLICATIONS

This application is a divisional of U.S. patent application Ser. No. 10/570,126, filed on Jun. 12, 2007, which is the National Stage of International Application Number PCT/GB04/03880, filed Sep. 10, 2004, which claims the benefit of U.S. Provisional Patent Application No. 60/502,514, filed Sep. 12, 2003, each of which is hereby incorporated by reference herein in its entirety, including any figures, tables, nucleic acid sequences, amino acid sequences, or drawings.

### FIELD OF THE INVENTION

The present invention relates to the preparation of cells for use in therapy. In particular, the present invention relates to the preparation of immortalised mammalian cells for therapeutic application.

### BACKGROUND TO THE INVENTION

SV40 is a small DNA tumour virus that elicits either a lytic infection in its natural host, macaque monkey cells, or neoplastic transformation in a wide variety of non-permissive rodent cells. One of the early gene products of SV40, the large T antigen, is a multifunctional phosphoprotein that has been studied extensively as a model system for understanding diverse and complex cellular phenomena such as nuclear transport, transcriptional regulation, eukaryotic DNA replication and deregulation of cell growth resulting in neoplastic transformation (reviewed in Fanning and Knippers, 1992; Manfredi and Prives, 1994; Sullivan and Pipas, 2002). It has even been suggested that SV40 may be a pathogen in a limited number of human tumours such as mesotheliomas and osteosarcomas (Gazdar et al., 2002).

T antigen's ability to deregulate cellular proliferation pathways is reflected in its very efficient immortalisation of primary rodent cells (Jat and Sharp, 1986; Petit et al., 1983), low frequency immortalisation of human cells (Shay et al., 1993), and transformation of established rodent cell lines to tumorigenicity (Brown et al., 1986). It alone is sufficient for immortalisation of rodent cells (Jat and Sharp, 1986) but requires additional activities such as reconstitution of telomerase activity for full immortalisation of human cells (Jha et al., 1998; O'Hare et al., 2001).

The ability of T antigen to deregulate cell proliferation mechanisms is dependent upon its specific interaction with a variety of host cell proteins. The study of T antigen associated proteins has led to the elucidation of many signalling pathways as well as identification of tumour suppressor genes (reviewed in (Manfredi and Prives, 1994; Sullivan and Pipas, 2002)). The tumour suppressor protein p53, mutated or deleted in the majority of human cancers, was originally discovered as a T antigen interacting protein (Lane and Crawford, 1979; Linzer and Levine, 1979). Identification of the interaction of T antigen with members of the Retinoblastoma (pRB) family of proteins has also resulted in substantial advances in understanding their normal cellular functions as cell cycle regulatory proteins (DeCaprio et al., 1988; Zalvide and DeCaprio, 1995). Additional T antigen binding partners such as p300 and p400 also contribute to the full repertoire of T antigen functions, but their precise roles remain unresolved (Eckner et al., 1996; Lill et al., 1997). More recently, it was found that the extreme N-terminus (amino acids 1-70) of T antigen constitutes a bona fide DnaJ domain (Campbell et al., 1997), which is required for efficient viral replication as well as some transformation functions (Campbell et al., 1997; Srinivasan et al., 1997; Stubdal et al., 1997; Sullivan and Pipas, 2002).

T antigen is clearly an extremely versatile viral oncoprotein. However, unlike the polyomavirus middle T antigen or activated Ras proteins, T antigen is a relatively weak transforming protein. Thus, even when T antigen expression is delivered by retroviral infection into almost every cell, only a few cells become transformed, suggesting that additional activities or genetic alterations may be required for full transformation (Jat and Sharp, 1986). Perhaps germane to the process of oncogenic transformation is the fact that expression of T antigen in human cells has been shown to cause genomic instability by inducing chromosomal aberrations and aneuploidy (Chang et al., 1997; Ray et al., 1990; Stewart and Bacchetti, 1991; Woods et al., 1994). While this function could be explained by disruption of p53 activity upon association with T antigen, the N-terminal 147 amino acids of the protein, which do not contain p53 binding sequences, can induce genome destabilisation as effectively as the wild-type protein (Woods et al., 1994). Destabilisation also does not absolutely require binding to pRB family members (Woods et al., 1994). The ability of T antigen to cause such karyotypic instability in human cells, has been found to correlate with its ability to deregulate normal mitotic checkpoints (Chang et al., 1997). The mechanism by which T antigen causes karyotypic instability or induces endoreduplication is not known.

### SUMMARY OF THE INVENTION

The present invention is based on the finding that T antigen binds to Bub1 protein kinase, and that this interaction is responsible for the genomic instability sometimes associated with T-antigen expressing cells. The interaction is not required for immortalisation of cells and so disrupting this binding is useful for the preparation of genetically stable immortalised cell lines.

According to a first aspect of the invention, there is a SV40 T antigen protein that lacks the ability to bind to the Bub1 protein.

According to a second aspect of the invention, a recombinant mammalian cell comprises a polynucleotide that encodes T antigen, wherein the expressed T antigen is modified to prevent binding to Bub1.

According to a third aspect of the present invention, a recombinant cell, as defined above, is used in therapy.

According to a fourth aspect of the present invention, a recombinant cell, as defined above, is used in the manufacture of a medicament for the treatment of a disorder associated with cell loss or cell damage.

### DESCRIPTION OF THE FIGURES

The present invention is described with reference to the accompanying drawings, wherein:

FIG. 1 illustrates various genetic constructs used in the two-hybrid analysis;

FIG. 2 illustrates the results of T antigen/Bub1 binding studies;

FIG. 3 illustrates the results of Bub1 kinase activity;

FIG. 4 illustrates the conserved sequence of the Bub1 binding domain in different T antigens and the immortalisation efficiency of different T antigen mutations;

FIG. 5 illustrates the results of the analysis of mitotic index;

FIG. 6 shows that chromosomal visualisation following immunostaining; and

FIG. 7 shows the cell cycle profile after DCB treatment.

### DESCRIPTION OF THE INVENTION

The present invention was identified by exploiting a yeast two-hybrid screen to search for cellular proteins that interact with the amino terminus of SV40 T antigen. It was shown that T antigen interacts specifically with the mitotic spindle assembly checkpoint protein, Bub1 (Hoyt et al., 1991). This interaction was confirmed by reciprocal co-immunoprecipitation analysis in a wide variety of cell types. Genetic analysis indicated that a specific tryptophan-containing motif on T antigen is required for its interaction with Bub1. Interaction with Bub1 is not required for immortalisation by T antigen but is necessary for transformation. T antigen expression results in a partial disruption of the spindle assembly checkpoint such that cells can undergo mitosis even in the presence of low levels of spindle damage. T antigen mutants that fail to interact with Bub1 are defective in their ability to modulate the spindle checkpoint.

The binding site for Bub1 on T antigen is distinct from the binding sites for previously reported T antigen interactors. The genetic analysis of Bub1 binding to T antigen indicates that determinants in the region of helix 4, between amino acids 89 and 97, are critical for the interaction. This segment of T antigen is located between the DnaJ domain (residues 1-70) and the “LXCXE” motif (residues 103-107) required for binding to pRB family members. The amino acid sequence for T antigen is shown in SEQ ID NO:1. The 89-97 deletion mutant (dl89-97) was the most defective mutant for binding Bub1, although each of the conserved tryptophans W91, W94 and W95 in the “WEXWW” motif were important for efficient binding (X refers to any amino acid). The deletion in mutant dl89-97 is unlikely to be grossly perturbing the T antigen structure, because dl89-97 T antigen has been shown to be able to alter the phosphorylation state of p130 as well as target it for degradation, indicating that the DnaJ domain and pocket protein binding functions are intact (Stubdal et al., 1997). Moreover, mutant dl89-97, as well as each of the tryptophan substitution mutants, was still capable of immortalizing rat embryo fibroblasts (REFs) as well as or better than wild-type T antigen. Dl89-97 T antigen can also bind to Hsc70, pRB and p53, activate an E2F transcriptional reporter like wild-type T antigen and successfully overcome a p53-dependent cell cycle arrest (Gjoerup et al., 2000). Finally, experiments carried out by the inventors with the actin assembly inhibitor DCB also support the conclusion that the dl89-97 mutant, like wild-type T antigen, overcomes the p53-dependent tetraploidy checkpoint (Andreassen et al., 2001; Lanni and Jacks, 1998; Minn et al., 1996). Thus, the overall structural integrity of dl89-97 T antigen is likely to be preserved. The “WEXWW” motif is conserved between SV40, BK, JC, and bovine polyomavirus T antigens. The degree of conservation between the viral T antigens suggests an important function for this motif, perhaps in a similar manner to the conserved “HPD(K/R)” motif of DnaJ domains.

T antigen enhances both the Bub1 autokinase activity as well as its activity towards exogenous substrates such as histone H1. This mode of action is quite unusual for T antigen. Usually, it inactivates its target proteins, which are often tumour suppressors. Since this activation was unusual, strenuous efforts were made to confirm that the kinase activity measured was truly due to Bub1. It was shown that the kinase signal can be silenced with specific siRNA oligos or DNA based RNAi directed against Bub1.

The interaction of Bub1 and T antigen may explain existing literature. Several reports have demonstrated that T antigen is able to induce aneuploidy and genetic instability, giving rise to both structural and numerical chromosome aberrations (Chang et al., 1997; Levine et al., 1991; Ray et al., 1990; Stewart and Bacchetti, 1991; Woods et al., 1994). It had been proposed originally, that this instability was most likely to be due to the ability of T antigen to interact with and inactivate the p53 protein. However, it was subsequently shown that an amino terminal 147 amino acid fragment of T antigen that is unable to interact with p53 can still efficiently induce instability, and that interaction with pRB was also not strictly required for the induction of genomic instability (Woods et al., 1994). Moreover, it was shown that loss of p53 in somatic cells did not result in aneuploidy, although a slight tendency towards tetraploidization was observed (Bunz et al., 2002). Thus, the mechanism by which T antigen causes karyotypic instability or induces endoreduplication is not known.

Since it had been shown that the amino terminus was sufficient to induce genome instability, a yeast two-hybrid screen was carried out using the N-terminal domain of T antigen as bait and a HeLa cDNA library as prey. One two-hybrid interactor thus identified was the human Bub1 protein kinase, a checkpoint protein involved in monitoring assembly of the mitotic spindle (Cahill et al., 1998; Roberts et al., 1994; Taylor and McKeon, 1997). The bub (budding uninhibited by benzimidazole) and mad (mitotic arrest deficient) genes were initially identified in yeast genetic screens (Hoyt et al., 1991; Li and Murray, 1991), and subsequently, mammalian counterparts were discovered. Current knowledge suggests that Bub1 together with Bub3 are components of a multi-protein spindle checkpoint complex at the kinetochore, which can sense lack of tension at the kinetochore and/or attachment of a microtubule to the kinetochore (Millband et al., 2002; Skoufias et al., 2001). Even if a single kinetochore is not attached, a signal is generated that is transduced through a signalling cascade and culminates in the inhibition of the ubiquitin ligase complex termed the anaphase promoting complex/cyclosome (APC/C) and a transient arrest of the cell cycle at the metaphase to anaphase transition. This checkpoint ensures the accurate segregation of sister chromatids at mitosis by monitoring their proper bivalent attachment to mitotic spindles. Bub1 has been found to be occasionally mutated in certain types of human cancer such as colorectal cancer, which is characterized by chromosomal instability and increased aneuploidy (Cahill et al., 1998; Ru et al., 2002; Shichiri et al., 2002).

The genetic analysis of T antigen mutants shown herein demonstrates that binding to Bub1 is dispensable for immortalization but may be required for transformation. Furthermore, T antigen, but not a non-Bub1 binding mutant of T, overrides the spindle checkpoint. The results suggest an important additional mechanism for the transforming activity of T antigen.

The inventors found that dl89-97 T antigen, which does not interact with Bub1, was defective for induction of endoreduplication in the presence of spindle damage.

The polynucleotide encoding T antigen may be comprised in a recombinant DNA or retroviral vector or construct to transduce/infect the cells. The vector or construct may further comprise a suitable promoter region to initiate transcription of DNA and a selectable marker which may be used to identify those cells that have undergone transduction/infection. Regulation of expression may be carried out by methods known to the skilled person. For example, regulation may be effected using the long terminal repeat (LTR) promoter. Alternative promoters will be apparent to the skilled person. For example, regulation may be effected using the cytomegalovirus (CMV) promoter. The CMV promoter is a very strong promoter, and may be preferred when the cells are neural cells, e.g. neuroepithelial stem cells.

The term “T antigen” refers to the large T antigen of the polyomavirus family. The term includes the natural and modified versions, including recombinant versions that retain the ability to immortalise a cell, but which can be regulated by external factors, e.g. temperature-sensitive mutants. The preferred T antigen is derived from SV40 virus.

A preferred embodiment is a temperature-sensitive mutant oncogene encoding a large T antigen. The large T antigen is preferred for cells that are to be used in transplantation therapies. The wild-type large T antigen requires components such as activated ras and small T antigen to become tumourigenic. Therefore, large T antigen on its own reduces the possibility of unconditional transformation. Using a temperature-sensitive mutant further reduces the ability of the antigen to induce uncontrolled transformation, although the use of a temperature-sensitive mutant is not a critical limitation of the present intention.

The mutant preferably comprises the nucleotide sequence encoding the large T antigen (SEQ ID No:1) having a U19 deletion, e.g. that corresponding to the early region of the U19 tsA58 double mutant; see U.S. Pat. No. 5,270,191. The mutant will however lack one or more of the amino acid residues indicated as 89-97 in SEQ ID No:1, or will comprise a mutation to one or more of said residues. The construction of this mutant is shown below. The U19 mutation prevents the antigen from binding to the SV40 origin of replication, ensuring that integrated proviruses that contain an origin of replication are safe. Methods for introducing suitable constructs into cells, are known to the skilled person.

The use of a temperature-sensitive mutant is desirable for the conditional immortalisation of cells. This has benefits for transplantation therapies where the cells can be maintained in culture conditions suitable for growth and replication, but when transplanted the unconditional growth is inhibited due to body temperature.

In use, it is preferred if the transduced or infected cell further comprises a polynucleotide encoding the catalytic sub-unit of the telomerase complex (hTERT); see International Patent Application Number WO-A-01/21790, the content of which is incorporated herein by reference. It has been found by the present inventors that the combination of T antigen comprising the Bub1 deletion and U19 deletion used in combination with hTERT results in karyotype stability and immortalisation. Cells having these components produce diploid cells that retain immortal character.

The T antigen and the polynucleotide encoding the telomerase may be comprised in a recombinant DNA or retroviral vector or construct to transduce/infect the cells. The two components may be incorporated into one vector (bicistronic) or each may be comprised in a separate vector. The vectors or constructs of the invention may further comprise a suitable promoter region to initiate transcription of DNA and a selectable marker which may be used to identify those cells that have undergone transduction/infection. Regulation of expression may be carried out by methods known to the skilled person. For example, regulation may be effected using the long terminal repeat (LTR) promoter, e.g. from Maloney Murine Leukemia virus. Alternative promoters will be apparent to the skilled person. For example, regulation may be effected using the cytomegalovirus (CMV) promoter. The CMV promoter is a very strong promoter, and may be preferred when the cells are neural cells, e.g. neuroepithelial stem cells.

A preferred (bicistronic) construct of the invention has the following order of elements:

LTR-hTERT-CMV promoter-TAg(U19tsA58)-IRES-neo-LTR.

The above construct lacks the Bub1 binding site in T antigen due to a deletion of amino acid residues 89-97. The LTR used in this construct is that from Maloney Murine Leukemia virus, IRES is the Internal Ribosome Entry Site and neo is the gene conferring G418 resistance when the gene is transfected into cells.

In a further preferred embodiment, the hTERT is provided in a separate construct, with the LTR as promoter, and a separate construct contains the T antigen (with Bub1 mutation).

Any mammalian cell may be used in the present invention.

For example, the cell may be an endothelial cell, and may be used for the revascularisation of the leg, heart and other organs. Preferably, the cell is a human somatic cell, e.g. human epithelial stem cell, which is capable of differentiation into a specific cell type. A particularly preferred cell is a human neuroepithelial stem cell which may be used in neural transplantation to repair cell loss or damage and correct behavioural or psychological deficits, e.g. Alzheimer's disease or Parkinson's disease. Alternatively, the cell may be a differentiated cell, e.g. the β cells of Islets of Langerhans. Additional cells include, but are not limited to, those obtainable from the endocrine glands, retinal cells, cochlear cells, liver cells, osteoblast and osteoclasts, myoblasts, fibroblasts and keratinocytes. Additional cells include stem cells or progenitor cells from the bone marrow, including stem cells that have mesodermal, endodermal and ectodermal cell types.

In a further preferred embodiment, the cell is a mammary cell, in particular a mammary luminal cell or a mammary fibroblast cell.

The transduced or infected cells may be cultured under conditions known to those skilled in the art. It is preferable that the cells are cultured under non-stressed conditions. A skilled person will appreciate the conditions suitable for each particular cell type, based on conventional culture techniques.

The transformed/immortalised cells according to the invention may be of use to create stable cell lines for therapeutic use. The cells may be of use in transplantation therapies, as will be appreciated by those skilled in the art.

The following experiments illustrate the invention.

### Yeast Two-Hybrid Analysis

The amino-terminal fragment of T antigen comprising nucleotides 1-408 was cloned in-frame with the LexA DNA-binding domain into pGilda, a galactose-inducible expression vector derived from pEG202 (Clontech). The MATα S. cerevisiae LEU2 selection strain EGY48 was transformed with the LexA-T antigen bait and the pJK103 LacZ reporter plasmid. The MATa S. cerevisiae strain RFY206 was transformed with a HeLa cDNA library (pJG4-5 library vector expressing B42 acid blob fusion proteins under the control of the GAL1 inducible promoter). The two strains were mated, and plated on selective media lacking leucine at 30° C. for 4 days to select for interactors. The colonies obtained were analysed for galactose-dependent growth on media lacking leucine, and galactose-dependent blue colour on media containing X-Gal. Plasmid DNA's from the strongest positives were recovered and transformed into E. coli JS4. Library plasmids were identified by colony PCR and classified by HaeIII restriction enzyme digestion patterns. Clones that interacted specifically with the LexA-T antigen bait were sequenced and identified by BLAST sequence comparisons with the GenBank and EMBL databases.

### Cells and Cell Culture

tsa8, tsa14, SV4, U2OS, 293T, Rat-1, NIH3T3, BOSC23 and Phoenix amphotropic cells were propagated by standard procedures (Gjoerup et al., 2000; Jat and Sharp, 1989). NIH3T3/T cells were derived by cotransfecting NIH 3T3 cells with pSE (encoding the SV40 early region) and the selectable plasmid pSV2neo. NIH3T3/V cells were derived by transfecting NIH3T3 cells with pKS (pBluescript, Stratagene) and with pSV2neo. Stable cell lines of U2OS cells were generated by cotransfection of wild-type or mutant T antigen expression vectors together with the puromycin resistance vector pE-puro (Gjoerup et al., 2000).

### Retroviral Infections and Immortalisation Assay

Retroviruses were prepared by transfection of pBabe-puro vectors into Phoenix amphotropic or BOSC23 ecotropic producer cells. Viral supernatants harvested 48 hours post-transfection were used for infection followed by subsequent selection of the infected cultures using 1.5 μg/ml puromycin. Immortalisation assays were carried out using cultures of secondary REF's as described previously. Representative dishes were stained after 14 days of selection and the number of colonies determined. At least 6 colonies were isolated for each mutant T antigen and expanded to determine if they could establish cell lines.

### Plasmids and Mutagenesis

The pSG5 expression vectors encoding wild-type T or mutant T antigens have been described previously (Gjoerup et al., 2000; Stubdal et al., 1997; Zalvide and DeCaprio, 1995). Recombinant retroviruses encoding wild-type T antigen or each of the mutants were prepared by inserting the cDNA into the pBabe-puro retroviral vector (Morgenstern and Land, 1990). Point mutants in the region between residues 89 and 97 in T antigen were generated by QuikChange site directed mutagenesis (Stratagene). A Bub1 expression vector was constructed by inserting HA-tagged full-length murine Bub1 (provided by J. van Deursen (Wang et al. 2001)) into pcDNA3.1 (Invitrogen). The kinase-dead mutant of mBub1 (K795A; (Kaplan et al., 2001; Sharp-Baker and Chen, 2001)) was generated by site directed mutagenesis.

The DNA based RNAi vector pSuper has been described (Brummelkamp et al., 2002). Duplex oligos [5′ GATCCCCGATGCATTTGAAGCCCAGTTTCAAGAGAACTGGGCTTCAAATGCATCTT TTTGGAAA 3′ (SEQ ID NO:2), 5′ AGCTTTTCCAAAAAGATG CATT TGAAGCCCAGTTCTCTTGAAACTGGGCTTCAAATGCATCGGG 3′ (SEQ ID NO:3] designed to target the same sequence that was successful for silencing Bub1 using siRNA oligos were inserted into pSuper and transfected into 293T cells.

### Silencing by siRNA Oligos

Oligos designed to target Bub1 essentially as recommended by Elbashir et al. (Elbashir et al., 2001) were obtained from Dharmacon (Lafayette, Colo.). The duplex oligos were “Bub1 sense” (5′ GAUGCAUUUGAAGCCCAGUdTdT 3′; SEQ ID NO:4) and “Bub1 anti-sense” (5′ ACUGG GCUUCAAAUGCAUCdTdT 3′; SEQ ID NO:5), targeting the region nt 1490 to 1512 relative to the human Bub1 ATG start codon.

### Microscopy

NIH3T3 cells were treated with nocodazole, 12 hours after plating from confluence, at a range of concentrations from 20-80 ng/ml for about 12 hours and 10 μg/ml of Hoechst 33258 for at least 2 hours and analyzed by microscopy.

### Flow Cytometry and Cell Synchronization

U2OS cells were treated with 100 ng/ml nocodazole for 48 hours, after which the cells were harvested in their medium, washed twice in ice cold PBS, fixed in ice cold 70% ethanol, stained with propidium iodide and analyzed by FACS (Gjoerup et al., 2000). For the DCB experiment, cells were treated with nocodazole, and mitotically arrested cells harvested by shake-off. These cells were then washed, replated in the presence of 10 μM DCB for 18 hours and analyzed by FACS as previously outlined (Andreassen et al., 2001).

### Antibodies, Immunoprecipitations and Western Blotting

Immunoprecipitation and Western blotting were carried out by standard procedures (Harlow and Lane, 1990). Briefly, cells were extracted with TEB lysis buffer [20 mM TrisHCl pH 7.5, 137 mM NaCl, 1% Nonidet P-40, 10% glycerol, 10 μg/ml leupeptin, 10 μg/ml pepstatin, 0.5 mM phenylmethylsulfonyl fluoride]. Nocodazole-treated NIH3T3 cells were harvested in their medium to avoid discarding cells in G2/M phase. 20-30 μg of total protein was analyzed by Western blotting using ECL (Amersham-Pharmacia). The antibodies used were: T antigen mouse monoclonal antibodies PAb416, PAb419, PAb423 and PAb100; Bub3 rabbit polyclonal antibody (obtained from P. Sorger); cyclin B1 (GNS1) mouse monoclonal antibody (Santa Cruz Biotechnology), securin rabbit polyclonal antibody (a gift from M. Kirschner). In co-immunoprecipitation experiments, Bub1 protein was visualized only after T antigen was immunoprecipitated with a monoclonal antibody recognizing the extreme C-terminus (PAb423). It was not detected when T antigen was immunoprecipitated with monoclonal antibodies PAb416, PAb419 or PAb100, probably due to reduced accessibility of the binding site. Our N-terminal and C-terminal Bub1 polyclonal antibodies, were raised in rabbits using the hBub1 amino acids 1-303 or 691-1085, respectively, fused in frame to glutathione S-transferase. The fusion proteins were insoluble, so inclusion bodies were purified and used for immunization by standard procedures (Harlow and Lane, 1990). In addition, Bub1 antibodies from other sources were used to analyse the T antigen/Bub1 interaction (Martinez-Exposito et al., 1999; Tang et al., 2001).

### Kinase Assays and V8 Phosphopeptide Mapping

For in vitro kinase assay, immunoprecipitations were carried out by standard protocols using an affinity-purified Bub1 polyclonal antibody (Tang et al., 2001). Cell extractions were performed with TEB including 2 mM EDTA. Protein A sepharose beads were washed twice with TEB and twice with kinase buffer [50 mM TrisHCl pH7.5, 10 mM MgCl₂, 1 mM NaF, 10 mM b-glycerophosphate, 1 mM DTT]. The in vitro kinase reaction was initiated by adding to the beads 30 μl of kinase buffer supplemented with 5 μCi γ[³²P] ATP and 20 μM cold ATP. V8 mapping was performed according to the basic outline originally reported by Cleveland et al. (Cleveland et al., 1977). The objective was to compare the ectopically expressed HA-mBub1 phosphopeptide pattern to that of endogenous mBub1 from NIH3T3/V or NIH3T3/T cells. In vitro kinase reactions were conducted with endogenous Bub1 from NIH3T3/V or NIH3T3/T cell lysates using Bub1 antibody in parallel with HA-mBub1 transfected U2OS cell lysates using the HA.11 monoclonal antibody directed against the HA tag (Covance). Reactions were resolved by SDS-PAGE and bands believed to correspond to either endogenous Bub1 or ectopically expressed HA-mBub1 were excised. The acrylamide pieces were carefully placed in the lanes of a second SDS-PAGE gel. Proteins within the gel slices were digested for 30 minutes when two thirds of the way through the stacking gel with either 10 or 50 ng of V8 protease (Sigma).

### Focus Formation Assay

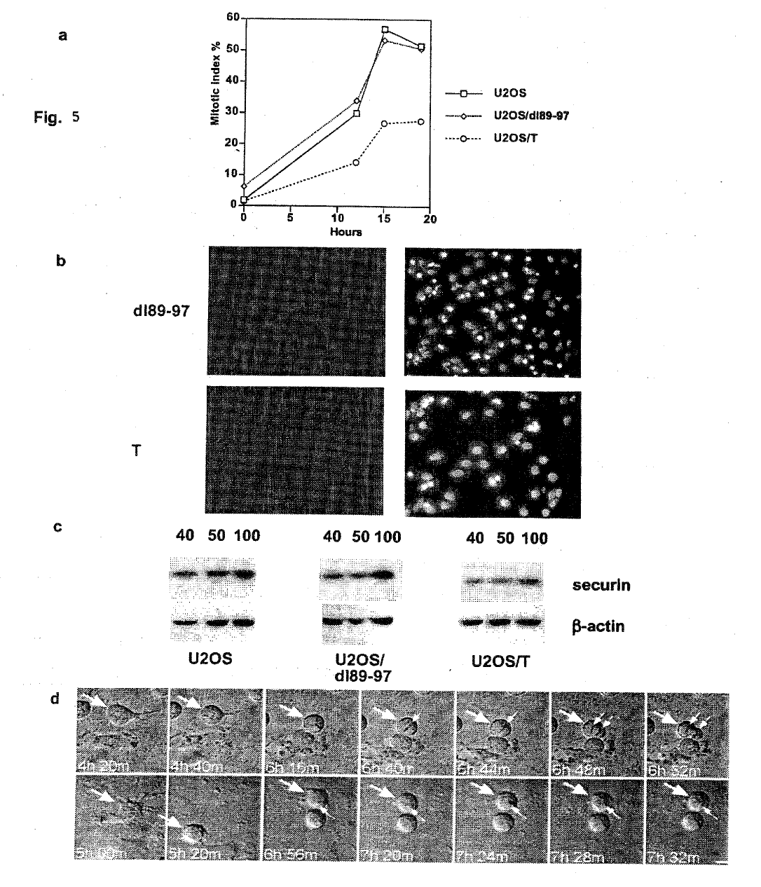

These were carried out on low passage Rat-1 cells using T antigen cDNA expression vectors as previously described (Kalderon and Smith, 1984). Foci were visualized by staining with 0.5% crystal violet approximately 3 weeks post-transfection. The assay was repeated 4-5 times with very similar outcomes to the experiment depicted in FIG. 5.

### Construction of Triple Mutants of Tag (U19tsA58dl89-97)

The mutant was constructed by fusing the front 400 nucleotides from a dl89-97 (i.e. lacking amino acids 89-97 as in SEQ ID No:1) large T cDNA to the remainder of the early region from the U19tsA58 mutant which is described in Almazan & McKay, Brain Res., 1992; 579(s): 234-245. U19tsA58 is a double mutant resulting from the fusion of U19 and tsA58. This construction was carried out using two plasmids pZipNeoSVU19tsA58 and pBabe dl89-97. The pZipNeoSVU19tsA58 carries the whole region of the double mutant inserted between the BamH1 sites. The insert corresponds to Bgl1 (nuc 5235) to Hpa1 (nuc 2666) inserted into the BamH1 site of pZipNeoSV(X)1 using BamH1 linkers after blunting.

The pBabe dl89-97 carries the cDNA for large T inserted into the BamH1—the insert is from an engineered Nco1 site at the ATG (approx nuc 5164 within SV40) to the BamH1 site (nuc2533) downstream of the poly A site.

The chimera was made by fusing a BamH1-Ear1 fragment (nt 4428 within SV40) from the dl89-97 plasmid (the front end of the cDNA for 89-97) to an Ear1-BamH1 fragment from pZipNeoSVU19tsA58. This large fragment supplied the remainder of the vector as well as the remainder of the SV40 early region. Due to the presence of multiple enzyme sites, the large fragment was constructed from two sections; Ear I to Dra III and Dra III to Bam H1. This resulted in the construction of a cDNA which has the front end from dl89-97 and the back end from the U19 (U19 mutation corresponds to two amino acid mutations within the middle fragment) tsA58 (single amino acid change within the c-terminus) and essentially a plasmid which is pZipNeoSVdl89-97U19tsA58 that represents a cDNA with the three mutations dl89-97, U19 and tsA58.

### Construction of hTERT/Bub1/U19 Mutant Cell Lines

### Summary of Cell Lines Made

6 different kinds of immortalised cell lines were made: 3 from human mammary fibroblasts and 3 from human mammary luminal cells as follows:

Human fibroblasts cell lines infected with the following virus constructs:
1. TAg(U19tsA58dl89-97)/hTERT (T Ag triple mutant). This cell line was called HMFts4.
2. TAg(U19dl89-97)/hTERT (T Ag double mutant, i.e. the T Ag was not temperature-sensitive). This cell line was called HMFU19.
3. TAg(U19tsA58)/hTERT (T Ag double mutant, i.e. there is no Bub1 mutation dl89-97). This cell line was called HMF 26.
Human mammary luminal cells infected with the following virus constructs:
4. T Ag (U19tsA58dl89-97)/hTERT (T Ag triple mutant). This cell line was called LTS4.
5. TAg(U19dl89-97)/hTERT (T Ag double mutant, i.e. the T Ag was not temperature-sensitive). This cell line was called LU19.
6. TAg(U19tsA58)/hTERT (T Ag double mutant, i.e. there is no Bub1 mutation dl89-97). This cell line was called L8/13.

### Materials and Methods for Generation of Cell Line Nos 1 and 3

The same general methodology was used for the different constructs to generate the other cell lines.

Generation of the T Ag (i.e. large T Antigen) triple mutant construct is as described above and this was used to produce a stable amphotrophic producer cell line using the teflya system. The amphotrophic virus from these cells was used to infect human mammary luminal cells and human mammary fibroblast cells in conjunction with a retrovirus expressing hTERT. The cells were doubly selected with G418 and hygromycin (via the neo gene on the triple mutant construct and the hygromycon-resistant gene on the hTERT construct). After drug selection the bulk transduced cells were maintained at 33° C., the permissive temperature for the ts T Ag and assayed for TRAP activity and tested in Western blots for T Ag expression.

These bulk cultures of infected cells were then subjected to several rounds of seeding at clonal density which involved removing the cells from the plate, subjecting them to a negative sort to remove any residual myoepithelial cells and then recloning the cells. The normal replicative potential of primary breast luminal cells is about 20 population doublings, which is approximately equal to one passage after seeding at clonal density. The inventors found previously that T Ag can extend cell lifespan by up to 80 population doublings (4-6 successive clonal passages). This allows a rapid depletion of the extended life span induced by T Ag alone and has allowed the inventors to determine if the cells expressing hTERT and Large T Ag and mutants thereof have exceeded this limit and are immortal.

At clone level 8 it was shown that T Ag and reconstitution of telomerase activity alone were not sufficient for immortalisation of these human cells whereas the combination of hTERT and T Ag readily immortalised them. These components can be provided separately from different viruses or in one step using the bicistronic retrovirus. The interaction of T Ag with Bub1 was not required for immortalization of the mammary luminal epithelial cells in conjunction with hTERT.

Following this long period of cultivation the cells were karyotyped to determine their karyotype stability i.e. to confirm that they were diploid or close to diploid.

### Results

### Yeast Two-Hybrid Analysis Identifies Bub1 as a Novel T Antigen Interactor

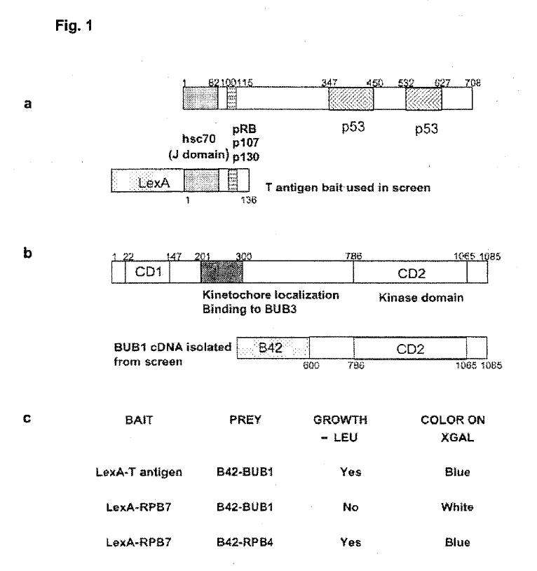

An amino-terminal fragment of large T antigen comprising nucleotides 1-408 (encoding amino acids 1-136 of T antigen) fused in-frame with the LexA DNA-binding domain was used as bait, as shown in FIG. 1a. The LexA-T antigen bait and a LacZ reporter plasmid were transformed into a MATα S. cerevisiae LEU2 selection strain. The resulting strain was mated with a MATa strain, which carried a HeLa cDNA library expressed as a fusion protein with the B42 transcriptional activator, and plated on selective media lacking leucine to select for interactors. An estimated 3×10⁵ diploid cells were screened and 122 LEU+ colonies collected after incubation at 30° C. for 4 days. Of these, 72 colonies showed galactose-dependent growth on media lacking leucine, and galactose-dependent blue color on media containing X-Gal. The 42 colonies with the strongest phenotype were subjected to sequencing and BLAST sequence comparisons with GenBank and EMBL databases. This showed that of the 42 colonies transformed and grown up, 39 were b-tubulin, 2 were a COPII vesicle coat protein and the third was a single clone of the carboxy terminus of Bub1 (encoding amino acids 600-1085), encompassing the protein's conserved kinase domain (FIG. 1b). Retransformation of clones representing each of these three interactors showed that only Bub1 was a true interactor.

### T Antigen and Bub1 Interact as Evidenced by Co-Immunoprecipitation

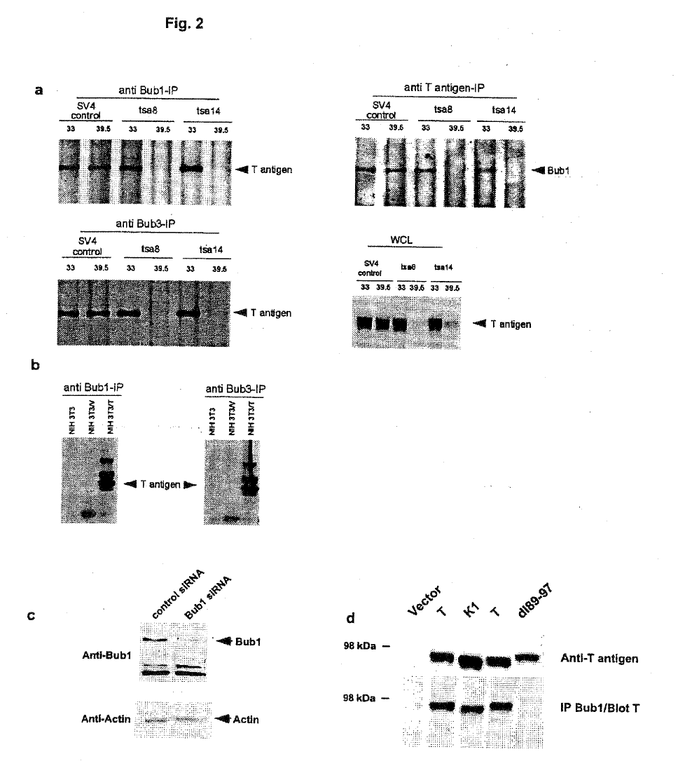

To confirm the yeast two-hybrid result, it was necessary to demonstrate the interaction of T antigen with Bub1 using full-length endogenous proteins from mammalian cell lysates prepared from a wide variety of cell lines such as rat tsa, mouse NIH3T3 and human U2OS cells. tsa8 and 14 cells are conditionally immortal cell lines derived by immortalizing rat embryo fibroblasts with the thermolabile tsA58 T antigen (Jat and Sharp, 1989). These cell lines grow continuously at the permissive temperature (33° C.), but undergo irreversible growth arrest in the G1 and G2 phases of the cell cycle upon shift to the non-permissive temperature (39.5° C.), where the tsA58 T antigen is rapidly inactivated (Jat and Sharp, 1989), FIG. 2a. T antigen and Bub1 could be reciprocally co-immunoprecipitated only at the permissive temperature from tsa cells (FIG. 2a). As a control for antibody affinity and stability of the protein-protein interaction at 33° C. and at 39.5° C., SV4 cells were used, which were derived by immortalization of rat embryo fibroblasts with wild-type T antigen and can proliferate continuously at both temperatures. T antigen and Bub1 interacted at both 33° C. and 39.5° C. in SV4 cells. Control immunoprecipitations with an irrelevant antibody demonstrated the co-immunoprecipitation was specific (data not shown). Direct immunoblotting showed that Bub1 was expressed at both temperatures, whereas T antigen was only present at 33° C. in the tsa cells (FIG. 2a).

T antigen also co-immunoprecipitated with Bub1 from NIH3T3 cells which ectopically express T antigen (NIH3T3/T) (FIG. 2b). As controls, parental NIH3T3 and NIH3T3 expressing an empty vector (NIH3T3/V) were used. We also examined the interaction in human U2OS cells stably expressing T antigen (U2OS/T). In these cells we first verified the specificity of the Bub1 antibody using siRNA technology. The level of endogenous Bub1 as judged by immunoblotting was significantly decreased upon transfection with a specific siRNA duplex targeting Bub1 (FIG. 2c). Subsequently, we showed that T antigen co-immunoprecipitates with Bub1 in U2OS/T cells (FIG. 2d). Taken together, these data show that Bub1 and T interact as assayed by co-immunoprecipitation using either Bub1 or T antigen antibody, and that the interaction can be detected in a wide variety of cell lines.

If T antigen and Bub1 are biological partners, T antigen might co-precipitate with other proteins known to complex with Bub1. Therefore, the interaction of T antigen with Bub3, another component of the spindle assembly checkpoint, was also tested. Bub3 is found in a complex with Bub1 in yeast, mouse and human cells (Hoyt et al., 1991; Martinez-Exposito et al., 1999; Roberts et al., 1994; Taylor et al., 1998). The binding of Bub3 to Bub1 is believed to be critical for the localization of Bub1 to the kinetochores of unattached chromosomes, and therefore for the proper assembly of the mitotic spindle (Taylor et al., 1998). It was found that T antigen co-immunoprecipitated with Bub3, both in tsa (FIG. 2a) and NIH3T3/T cells (FIG. 2b), suggesting that T antigen and Bub1-Bub3 are components of the same protein complex.

### T Antigen Enhances the Bub1 Kinase Activity

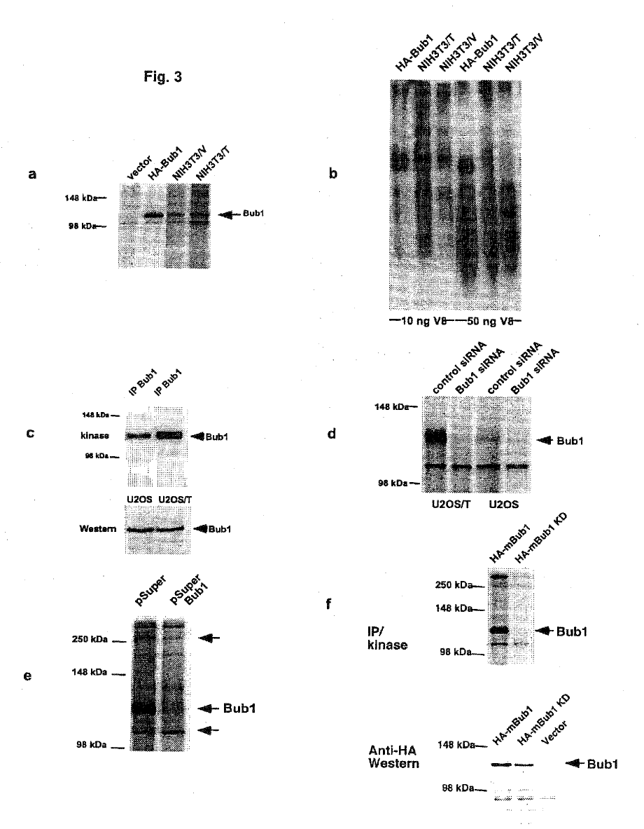

Further experiments were carried out to examine whether T antigen has any biochemical effect on Bub1. To this end, the kinase activity of Bub1 was investigated. Probably due to the low endogenous kinase activity of Bub1, most previous studies have focussed on purified recombinant Bub1or ectopically expressed epitope-tagged protein (Kaplan et al., 2001; Martinez-Exposito et al., 1999; Taylor et al., 1998). However, it was decided to look at the physiological level of Bub1 kinase activity. Initially, the endogenous Bub1 kinase activity was studied comparing NIH3T3/V and NIH3T3/T cells (FIG. 3a). Cell lysates were immunoprecipitated with anti-Bub1 antibody and in vitro kinase assays were performed. For comparison, ectopic HA-mBub1 immunoprecipitated with anti-HA antibody from U2OS cells transiently transfected with HA-mBub1 expression construct was also included. With normalized amounts of total protein used for immunoprecipitation, the Bub1 autokinase signal was always stronger from NIH3T3/T than NIH3T3/V cells. Furthermore, the band comigrated with ectopic HA-mBub1. However, as no previous report has demonstrated endogenous Bub1 kinase activity, it was necessary to verify that the band detected was authentic Bub1. Therefore, S. aureus V8 protease phosphopeptide mapping was carried out to compare the digestion pattern of putative endogenous mBub1 with that of ectopic HA-mBub1. The close degree of similarity between V8 digestion patterns strongly suggests that the band indeed represents endogenous Bub1 (FIG. 3b). When examining the in vitro kinase activity of endogenous Bub1 towards an exogenous substrate, histone H1, it was found that the activity was also higher in NIH3T3/T versus NIH3T3/V cells (data not shown).

Since the Bub1 auto-kinase signal was repeatedly cleaner and more robust in U2OS cells than in NIH3T3, further analysis was focused on this cell system. Interestingly, in vitro kinase assays performed with normalized amounts of total protein and using Bub1 antibody (Tang et al., 2001), indicated that the Bub1 autokinase activity was enhanced in U2OS/T versus U2OS (FIG. 3c). Western blotting demonstrated equal levels of Bub1 in U2OS compared to U2OS/T (FIG. 3c).

To confirm that the signal represented endogenous Bub1 kinase activity, siRNA technology was used. As shown in FIG. 3d, transfection of a Bub1 specific siRNA for 48 hours virtually completely eliminated the Bub1 autokinase signal from either U2OS or U2OS/T cells, whereas the control siRNA had no effect. In accordance with FIG. 3c, the Bub1 autokinase activity was elevated in U2OS/T compared with U2OS. As additional confirmation of the band being dependent on Bub1 expression, we transfected a DNA-based RNAi vector, pSuper (Brummelkamp et al., 2002) which targets the same region of the Bub1 mRNA as the siRNA oligos. These experiments were conducted in human 293T cells that are highly transfectable and express T antigen, thus ensuring that the Bub1 autokinase signal was measurable. It was evident that the Bub1 autokinase signal was substantially reduced in cells transfected with a DNA-based Bub1 RNAi vector (‘pSuper Bub1’) when compared with the empty vector (‘pSuper’) (FIG. 3e). Moreover, two other bands of approximately 105 kDa and 300 kDa were reduced in intensity upon silencing of Bub1 kinase activity. These two bands represent potential cellular targets of the Bub1 kinase.

Since it was formally possible that phosphorylation of Bub1 in the in vitro kinase assays was due to a coprecipitating kinase, experiments were undertaken to address this possibility. U2OS cells were transfected with either the HA-tagged mBub1 expression vector (Wang et al., 2001), or a vector encoding an HA-tagged mBub1 carrying a mutation in the critical lysine of the ATP binding motif (mBub1 K795A), hence predicted to be kinase defective. As shown in FIG. 3f, anti-HA immunoprecipitates from the HA-mBub1 transfectant displayed abundant Bub1 in vitro kinase activity. In marked contrast, the HA-mBub1 K795A mutant (labelled ‘KD’ in FIG. 3f) proved to be truly kinase defective as predicted, since the autokinase signal disappeared. A Western blot with anti-HA antibody demonstrated that the wild-type and kinase defective mBub1 were expressed at similar levels (FIG. 3f, bottom). Taken together, these experiments suggest that most of the signal corresponding to Bub1 in in vitro kinase reactions was due to autophosphorylation, rather than its phosphorylation by an associated kinase, and that T antigen expression results in stimulation of its kinase activity.

### Genetic Analysis of T Antigen/Bub1 Complex Formation

To identify the Bub1 binding site on T antigen, mutants were used that have previously been isolated within the amino terminal 136 amino acids. Initial experiments were conducted with U2OS lines stably expressing T antigen or various mutants. As shown in FIG. 2d, a deletion mutant of T antigen, dl89-97 (Stubdal et al., 1997), failed to bind Bub1 in a co-immunoprecipitation assay, while wild-type T antigen showed significant binding. The T antigen mutant K1 (Kalderon and Smith, 1984), which fails to bind pRB family members, retained binding to Bub1 (FIG. 2d). Similarly, the D44N mutant carrying a defective DnaJ domain (Campbell et al., 1997), also retained Bub1 binding (data not shown).

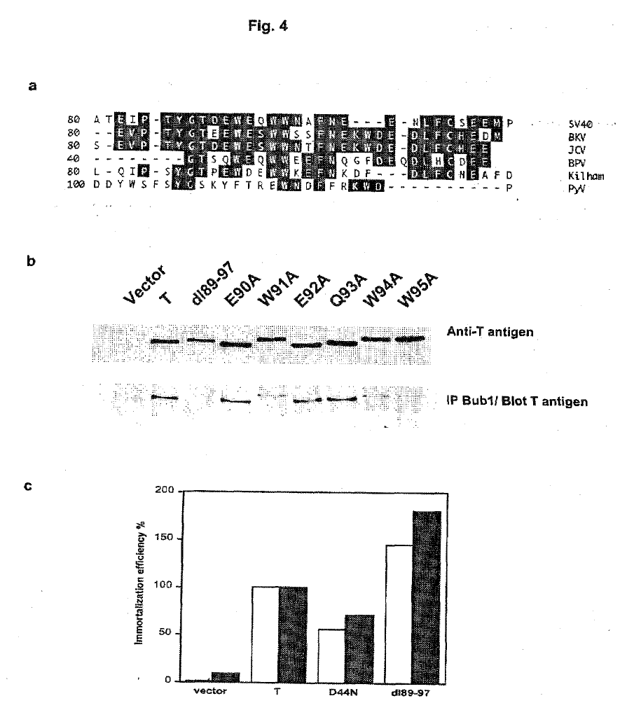

These analyses demonstrated that the binding between Bub1 and T antigen required one or more amino acids within residues 89-97 of T antigen. To more precisely map the interaction site, point mutants were generated. Selection of the specific residues of T antigen to mutate was guided by their conservation between different polyomavirus family members. As shown in FIG. 4a, the motif “WEXWW” was conserved between all large T antigens with the exception of mouse polyomavirus T antigen. We concentrated our efforts on mutating every residue of the conserved motif, as well as neighbouring amino acids, to alanine by site directed mutagenesis (alanine scanning mutagenesis). Hence, mutants E90A, W91A, E92A, Q93A, W94A and W95A were generated for testing in binding assays. pBabe-puro vectors for expression of the mutants were prepared, packaged into retroviruses and used to infect Rat-1 cells for production of pools of stable cell lines after puromycin selection. Each culture was lysed and immunoprecipitated with Bub1 antibody followed by immunoblotting with T antigen antibody. Whole cell lysates were analyzed in parallel to assess the amount of T antigen used for the immunoprecipitation. As shown in FIG. 4b, the co-immunoprecipitation analysis revealed that in Rat-1 cells T antigen also associated with Bub1, and that the deletion mutant dl89-97 was defective for this interaction consistent with our previous data in U2OS cells (FIG. 2d). Interestingly, each of the tryptophan substitution mutants (W91A, W94A, and W95A) was partially defective for binding Bub1, whereas mutants E90A, E92A and Q93A were able to bind Bub1. This result emphasizes the key role played by the conserved tryptophan residues within the “WEXWW” motif for binding Bub1. Perhaps not surprisingly, the single tryptophan substitution mutants retained some binding, whereas the much broader deletion mutant dl89-97 was almost totally defective. Interestingly, the T antigens encoded by the tryptophan substitution mutants, like the dl89-97 mutant, displayed an aberrant, more retarded mobility on SDS-PAGE that could be due to changes in modification.

### Bub1 Binding is Dispensable for T Antigen Mediated Immortalization

The immortalization potential of selected T antigen mutants was determined by retroviral infection of secondary rat embryo fibroblasts, followed by puromycin selection. After 14 days of puromycin selection, representative dishes were stained and counted to determine the immortalization efficiency of each mutant. At least 6 colonies were isolated for each and expanded to determine whether they would establish cell lines. The results for colony formation efficiency for two independent experiments depicted in FIG. 4c show that both the D44N and the dl89-97 mutant readily formed colonies. In addition, colonies isolated for each of these mutants readily established cell lines that could be serially subcultured demonstrating that they are both able to immortalize rat embryo fibroblasts. The dl89-97 mutant was repeatedly slightly more efficient than wild-type T antigen in colony formation (FIG. 4c). Furthermore, all of the point mutants were as efficient or often better than wild-type T antigen when tested for rat embryo fibroblast immortalization (data not shown). Taken together, this demonstrates that the dl89-97 mutant as well as the point mutants are not universally defective, as would be expected if their structure was globally disrupted.

### Genetic Analysis Suggests that the Interaction of T Antigen with Bub1 is Required for Transformation

Since Bub1 mutations had previously been identified in certain human cancers, it was desirable to test if interaction of Bub1 with T antigen might contribute to its transforming activity. One assay that measures T antigen transforming activity is based on its ability to overcome contact inhibition and form dense foci in Rat-1 cells (Kalderon and Smith, 1984). Hence, a cDNA expression vector encoding wild-type T antigen, deletion mutant dl89-97, or each of the point mutants, was transfected into Rat-1 cells. Three weeks later dense foci were visualized by staining with crystal violet. Strikingly, the mutant dl89-97 as well as each of the tryptophan substitution mutants W91A, W94A and W95A were severely defective for focus formation, whereas mutants E90A and E92A showed a partial defect, and mutant Q93A was unaffected relative to wild-type T antigen. These results correlate with the Bub1 binding data, where dl89-97, W91A, W94A and W95A have the most severe binding defect and Q93A the least, if any at all. This suggests that interaction with Bub1 may be required for efficient T antigen induced focus formation in Rat-1 cells.

### T Antigen Compromises the Spindle Checkpoint

Progression through the spindle assembly checkpoint is dependent upon the presence of a functional mitotic spindle. Disruption of the spindle by microtubule-depolymerizing drugs such as nocodazole causes the cells to arrest in mitosis (Li and Benezra, 1996; Taylor and McKeon, 1997). If the interaction between T antigen, Bub1 and Bub3 is functional, T antigen expression might alter the normal response of the cells to nocodazole. In fact, a hallmark of mitotic spindle checkpoint defective cells is a failure to arrest in response to microtubule-depolymerizing drugs (Li and Benezra, 1996; Taylor and McKeon, 1997). To test the response of cells to nocodazole the mitotic index of U2OS, U2OS expressing T or the dl89-97 mutant was examined after treatment with 50 ng/ml nocodazole for 12, 15 or 19 hours. The mitotic index was calculated by counting the proportion of cells with condensed chromatin after visualizing the DNA using Hoechst 33342 staining. As shown in the graph in FIG. 5a, the mitotic index in T antigen expressing cells was at all time points significantly lower than that of U2OS or U2OS expressing the dl89-97 mutant. To confirm these results, the mitotic index was also determined by staining the cells with antibody to phospho-histone H3, a marker of mitotic cells. After counting non-mitotic cells by visualizing their DNA with DAPI staining, the proportion of phospho-histone H3 positive cells was then calculated. Based on this approach, 39.9% of the U2OS cells were mitotic, versus 40.8% of the U2OS/dl89-97 and only 14.6% of the U2OS/T, when the cells were challenged with nocodazole for 15 hours. A representative field of phospho-histone H3 positive cells is shown in FIG. 5b for either U2OS/T or U2OS/dl89-97. Both assays for measuring the mitotic index, that is, staining with Hoechst or for phospho-histone H3, yielded very similar results and indicated that wild-type T antigen, but not the Bub1 binding mutant dl89-97, substantially decreased the mitotic index by overriding the checkpoint. Bypass of the spindle checkpoint should be accompanied by increased endoreduplication in the presence of nocodazole (Taylor and McKeon, 1997). As shown in Table 1, we observed that 45.7% of the U2OS/T cells underwent endoreduplication (>4N DNA content), whereas in U2OS and U2OS/dl89-97 cells only 24.8% and 20.6% underwent endoreduplication after nocodazole treatment.

TABLE 1

| Cell line | % >4N cells |
|---|---|
| U2OS | 14.1 |
| U2OS, 100 ng/ml nocodazole | 24.8 |
| U2OS/dl89-97, 100 ng/ml nocadazole | 20.6 |
| U2OS/T, 100 ng/ml nocodazole | 45.7 |

Hence, the decreased mitotic index and increased propensity for endoreduplication are both consistent with a T antigen induced override of the spindle checkpoint dependent on Bub1 binding. Yet another measure of spindle checkpoint dysfunction would be premature degradation of securin (Michel et al., 2001), the inhibitor of sister-chromatid separation targeted by the spindle checkpoint. Cells were synchronized at the G1/S border by mimosine treatment, released for 6 hours and then exposed to 40, 50 or 100 ng/ml of nocodazole for 12 hours to trigger the spindle checkpoint. The U2OS and U2OS/dl89-97 cells exhibited a gradual increase in securin levels with increasing nocodazole concentration, whereas the U2OS/T cells contained low levels of securin at all the nocodazole concentrations tested (FIG. 5c).

To carefully study and compare the temporal response of rat embryo fibroblasts immortalized either with wild-type T antigen or the dl89-97 mutant to low levels of spindle damage, time-lapse interference microscopy was performed. In a dose-response experiment, it was found that 24 ng/ml of nocodazole differentially affected T-versus dl89-97-expressing cells. At this concentration of nocodazole, it was observed that approximately 90% of the T expressing cells initially round up and then proceed to anaphase; the fate of the remaining 10% remains uncertain. The vast majority of the cells that reach anaphase subsequently reattached to the bottom of the culture dish without completing cytokinesis, presumably because the microtubules are depolymerized. In contrast, >95% of the dl89-97-expressing cells initially round up but they eventually flatten out without reaching anaphase. The temporal nocodazole response of a representative wild-type T- or dl89-97-expressing cell is illustrated in FIG. 5d. The observation that wild-type T- and dl89-97-expressing cells respond differently to this concentration of the spindle toxin strongly supports the notion that wild-type T, but not the Bub1-binding mutant, overrides the spindle checkpoint.

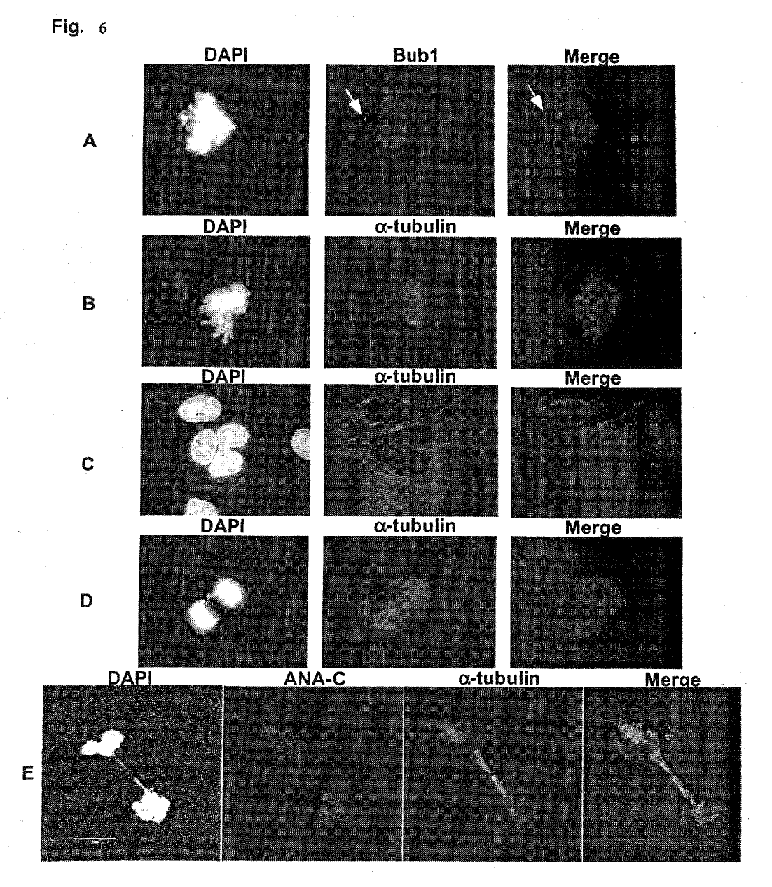

Since a failure to arrest at the spindle checkpoint would result in chromosome mis-segregation, the U2OS/T cells were examined for appearance of lagging chromosomes, anaphase chromatin bridges and multinucleated cells, all of which would indicate a faulty checkpoint. All of these mitotic abnormalities were observed in the T expressing cells; representative examples of chromatin bridges (FIGS. 6D and E), a multi-nucleated cell (FIG. 6C) and lagging metaphase chromosomes (FIGS. 6A and B) are shown in FIG. 6. Chromosomal DNA was visualized by DAPI staining, whereas the spindle was observed following anti-a-tubulin immunostaining. To visualize the centromere or kinetochore, immunofluorescence was conducted with either ANA-C anti-centromere human auto-immune serum (FIG. 6E) or anti-Bub1 antibody (FIG. 6A). The presence of anaphase bridges, lagging chromosomes and multi-nucleated cells are all consistent with a defective checkpoint.

Because T antigen is a multifunctional protein, it was essential to demonstrate that disruption of the spindle checkpoint by T antigen is linked to Bub1 binding and not to other functions of T, especially the binding of p53.

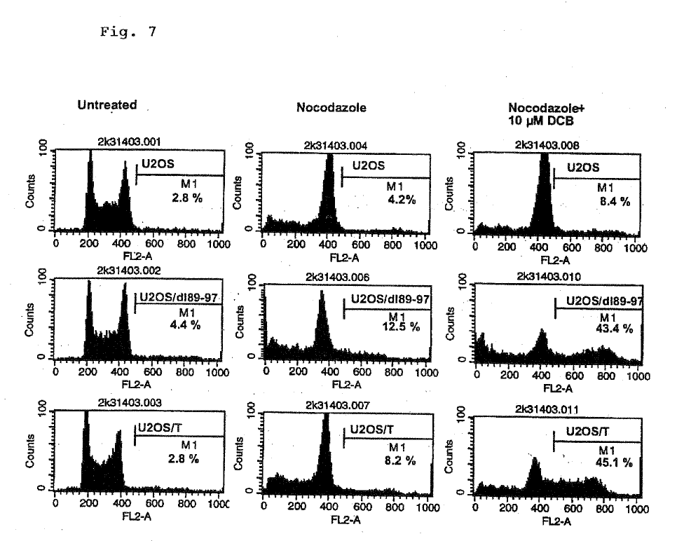

Although p53 has been demonstrated not to be required for the spindle assembly checkpoint per se, it has been clearly shown to be required for a postmitotic checkpoint that appears to monitor tetraploidy (Andreassen et al., 2001; Lanni and Jacks, 1998; Minn et al., 1996). When cells are challenged with spindle damage, they arrest only transiently at the metaphase to anaphase transition. Ultimately, a cell escapes arrest by a process termed “mitotic slippage” and subsequently encounters the tetraploidy checkpoint. Hence, cells with wild-type p53 arrest with 4N DNA content in a “G1 like” state after prolonged nocodazole treatment, whereas cells without p53 undergo endoreduplication. To rule out the possibility that the dl89-97 mutant is defective in overcoming the tetraploidy checkpoint, the cells were treated with dihydrocytochalasin B (DCB), which is an inhibitor of cytokinesis that exclusively triggers the p53-dependent tetraploidy checkpoint but not the spindle assembly checkpoint (Andreassen et al., 2001). The cells were first synchronized using a mitotic shake-off after nocodazole treatment and then released into 10 μM DCB. FIG. 7 shows the cell cycle profile of untreated as well as mitotically synchronized (mitotic shake-off) U2OS, U2OS/dl89-97 and U2OS/T. As expected, when the U2OS cells were released into DCB, they remained arrested with 4N DNA content since they have wild-type p53. Strikingly, both the U2OS/dl89-97 and the U2OS/T cells underwent significant and comparable endoreduplication (43.4% and 45.1% within the M1 gate, respectively). Taken together, this demonstrates clearly that the dl89-97 mutant is capable of functionally inactivating p53, and that its defect is likely to be linked to lack of Bub1 binding.

### Karyotype Stability

### Cell Line No. 1 (HMF TS4).

These are fibroblast cells infected with 2 separate viruses: T Ag(U19dl89-97tsA58) and hTERT.

The modal number of chromosomes was 46, i.e. diploid. The chromosome range was 33-59. The counts of 13 cells were 33, 40, 43, 44, 45, 46, 46, 46, 46, 46, 47, 59. One double minute and 2 small chromosome fragments were detected in one out of thirteen cells.

### Cell Line No. 2 (HMF U19)

These are human mammary fibroblasts infected with two separate viruses for hTERT and T Ag(U19dl89-97). This is the non-temperature-sensitive, non-DNA binding, non-Bub1 binding T Ag. Modal number of chromosomes was 44. Chromosome range was 29-44. The counts of ten cells were diploid with a variable chromosome number. However, some chromosomes may have been lost during the chromosome preparation.

### Cell Line No. 3 (HMF 26)

This human mammary fibroblast cell line containing the biscistronic virus expressed intermediate level of T antigen and appropriate level of hTERT. The HMF 26 cells are hypertriploid (aneuploid) with modal numbers of chromosomes: 71-73. The chromosome range was 69-78. The counts of ten cells were 69, 71, 71, 71, 73, 73, 73, 76, 76, 78. Chromosome fragments were detected in eight out of ten cells. These data indicate numerical and structural chromosomal abnormalities.

### Cell Line No. 4 (LTS4)

These are luminal cells infected with 2 separate viruses: T Ag(U19dl89-97tsA58) and hTERT.

The chromosome range of LTS4 cells was 36-51. The modal number is 51. The counts of six cells were 36, 37, 38, 48, 51, 51. No chromosome fragments or constrictions were detected. The cells are close to diploid (where n=46).

### Cell Line No. 5 (LU19)

Luminal cells infected with two separate viruses: hTERT and T Ag(U19dl89-97). The modal number of chromosomes was 46 ie the cells are diploid. The chromosome range of LU19 cells was 46-93. The counts of 11 cells were 46, 46, 46, 46, 48, 48, 50, 51, 55, 77, 93. There were no chromosome abnormalities detected in three diploid chromosome LU19 cells.

Chromosome constrictions were detected in three cells. Two double minutes were present in one cell (image/12). Small chromosome fragments were detected in two cells. Two dicentric chromosomes were found in one cell.

### Cell Line No. 6 (L8/13)

Luminal cells infected with T Ag(U19tsA58) and hTERT driven from two separate viruses. The modal number of chromosomes in L8/13 cells could not be determined. The chromosome range was 46-129 indicating aneuploidy. The counts of seven cells were 46, 48, 60, 62, 66, 98, 129. The data indicate numerical chromosome abnormalities on most cells. Two chromosome fragments were detected in one of these cells.

In summary, it was found that the cells transformed with T antigen having the deletion of U19 and the Bub1 binding site, together with the catalytic sub-unit of the telomerase complex (hTERT) retained karyotype stability and immortalised character. In particular the construct having the following order of elements would provide the best balance between expression levels, immortalisation and karyotype stability:

LTR-hTERT-CMV promoter-TAg(U19tsA58dl89-97)-IRES-neo-LTR

Karyotype stability and immortalisation is not dependent on whether TAg is temperature-sensitive or not and whether the components are provided in a bicistronic virus or in separate viruses.

### REFERENCES

Andreassen, P. R., Lohez, O. D., Lacroix, F. B., and Margolis, R. L. (2001). Tetraploid state induces p53-dependent arrest of nontransformed mammalian cells in G1. Mol Biol Cell 12, 1315-1328.
Brown, M., McCormack, M., Zinn, K. G., Farrell, M. P., Bikel, I., and Livingston, D. M. (1986). A recombinant murine retrovirus for simian virus 40 large T cDNA transforms mouse fibroblasts to anchorage-independent growth. J Virol 60, 290-293.
Brummelkamp, T. R., Bernards, R., and Agami, R. (2002). A system for stable expression of short interfering RNAs in mammalian cells. Science 296, 550-553.
Bunz, F., Fauth, C., Speicher, M. R., Dutriaux, A., Sedivy, J. M., Kinzler, K. W., Vogelstein, B., and Lengauer, C. (2002). Targeted inactivation of p53 in human cells does not result in aneuploidy. Cancer Res 62, 1129-1133.
Cahill, D. P., Lengauer, C., Yu, J., Riggins, G. J., Willson, J. K., Markowitz, S. D., Kinzler, K. W., and Vogelstein, B. (1998). Mutations of mitotic checkpoint genes in human cancers. Nature 392, 300-303.
Campbell, K. S., Mullane, K. P., Aksoy, I. A., Stubdal, H., Zalvide, J., Pipas, J. M., Silver, P. A., Roberts, T. M., Schaffhausen, B. S., and DeCaprio, J. A. (1997). DnaJ/hsp40 chaperone domain of SV40 large T antigen promotes efficient viral DNA replication. Genes Dev 11, 1098-1110.
Chang, T. H., Ray, F. A., Thompson, D. A., and Schlegel, R. (1997). Disregulation of mitotic checkpoints and regulatory proteins following acute expression of SV40 large T antigen in diploid human cells. Oncogene 14, 2383-2393.
Cleveland, D. W., Fischer, S. G., Kirschner, M. W., and Laemmli, U. K. (1977). Peptide mapping by limited proteolysis in sodium dodecyl sulfate and analysis by gel electrophoresis. J Biol Chem 252, 1102-1106.
DeCaprio, J. A., Ludlow, J. W., Figge, J., Shew, J. Y., Huang, C. M., Lee, W. H., Marsilio, E., Paucha, E., and Livingston, D. M. (1988). SV40 large tumour antigen forms a specific complex with the product of the retinoblastoma susceptibility gene. Cell 54, 275-283.
Eckner, R., Ludlow, J. W., Lill, N. L., Oldread, E., Arany, Z., Modjtahedi, N., DeCaprio, J. A., Livingston, D. M., and Morgan, J. A. (1996). Association of p300 and CBP with simian virus 40 large T antigen. Mol Cell Biol 16, 3454-3464.
Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498.
Fanning, E., and Knippers, R. (1992). Structure and function of simian virus 40 large tumour antigen. Annu Rev Biochem 61, 55-85.
Friedrich, T. D., Laffin, J., and Lehman, J. M. (1992). Simian virus 40 large T-antigen function is required for induction of tetraploid DNA content during lytic infection. J Virol 66, 4576-4579.
Gazdar, A. F., Butel, J. S., and Carbone, M. (2002). Opinion: SV40 and human tumours: myth, association or causality? Nat Rev Cancer 2, 957-964.
Gjoerup, O., Chao, H., DeCaprio, J. A., and Roberts, T. M. (2000). pRB-dependent, J domain-independent function of simian virus 40 large T antigen in override of p53 growth suppression. J Virol 74, 864-874.
Harlow, E., and Lane, D. (1990). Antibodies: A Laboratory Manual, Cold Spring Harbor Laboratory Press).
Hoyt, M. A., Totis, L., and Roberts, B. T. (1991). S. cerevisiae genes required for cell cycle arrest in response to loss of microtubule function. Cell 66, 507-517.
Jat, P. S., and Sharp, P. A. (1986). Large T antigens of simian virus 40 and polyomavirus efficiently establish primary fibroblasts. J Virol 59, 746-750.
Jat, P. S., and Sharp, P. A. (1989). Cell lines established by a temperature-sensitive simian virus 40 large-T-antigen gene are growth restricted at the nonpermissive temperature. Mol Cell Biol 9, 1672-1681.
Jha, K. K., Banga, S., Palejwala, V., and Ozer, H. L. (1998). SV40-Mediated immortalisation. Exp Cell Res 245, 1-7.
Jin, D. Y., Spencer, F., and Jeang, K. T. (1998). Human T cell leukemia virus type 1 oncoprotein Tax targets the human mitotic checkpoint protein MAD1. Cell 93, 81-91.
Kalderon, D., and Smith, A. E. (1984). In vitro mutagenesis of a putative DNA binding domain of SV40 large-T. Virology 139, 109-137.
Kaplan, K. B., Burds, A. A., Swedlow, J. R., Bekir, S. S., Sorger, P. K., and Nathke, I. S. (2001). A role for the Adenomatous Polyposis Coli protein in chromosome segregation. Nat Cell Biol 3, 429-432.
Lane, D. P., and Crawford, L. V. (1979). T antigen is bound to a host protein in SV40-transformed cells. Nature 278, 261-263.
Lanni, J. S., and Jacks, T. (1998). Characterization of the p53-dependent postmitotic checkpoint following spindle disruption. Mol Cell Biol 18, 1055-1064.
Lee, H., Trainer, A. H., Friedman, L. S., Thistlethwaite, F. C., Evans, M. J., Ponder, B. A., and Venkitaraman, A. R. (1999). Mitotic checkpoint inactivation fosters transformation in cells lacking the breast cancer susceptibility gene, Brca2. Mol Cell 4, 1-10.
Levine, D. S., Sanchez, C. A., Rabinovitch, P. S., and Reid, B. J. (1991). Formation of the tetraploid intermediate is associated with the development of cells with more than four centrioles in the elastase-simian virus 40 tumour antigen transgenic mouse model of pancreatic cancer. Proc Natl Acad Sci USA 88, 6427-6431.
Li, R., and Murray, A. W. (1991). Feedback control of mitosis in budding yeast. Cell 66, 519-531.
Li, Y., and Benezra, R. (1996). Identification of a human mitotic checkpoint gene: hsMAD2. Science 274, 246-248.
Lill, N. L., Tevethia, M. J., Eckner, R., Livingston, D. M., and Modjtahedi, N. (1997). p300 family members associate with the carboxyl terminus of simian virus 40 large tumour antigen. J Virol 71, 129-137.
Linzer, D. I., and Levine, A. J. (1979). Characterization of a 54K dalton cellular SV40 tumour antigen present in SV40-transformed cells and uninfected embryonal carcinoma cells. Cell 17, 43-52.
Manfredi, J. J., and Prives, C. (1994). The transforming activity of simian virus 40 large tumour antigen. Biochim Biophys Acta 1198, 65-83.
Martinez-Exposito, M. J., Kaplan, K. B., Copeland, J., and Sorger, P. K. (1999). Retention of the BUB3 checkpoint protein on lagging chromosomes. Proc Natl Acad Sci USA 96, 8493-8498.
Michel, L. S., Liberal, V., Chatterjee, A., Kirchwegger, R., Pasche, B., Gerald, W., Dobles, M., Sorger, P. K., Murty, V. V., and Benezra, R. (2001). MAD2 haplo-insufficiency causes premature anaphase and chromosome instability in mammalian cells. Nature 409, 355-359.
Millband, D. N., Campbell, L., and Hardwick, K. G. (2002). The awesome power of multiple model systems: interpreting the complex nature of spindle checkpoint signalling. Trends Cell Biol 12, 205-209.
Minn, A. J., Boise, L. H., and Thompson, C. B. (1996). Expression of BcI-xL and loss of p53 can cooperate to overcome a cell cycle checkpoint induced by mitotic spindle damage. Genes Dev 10, 2621-2631.
Morgenstern, J. P., and Land, H. (1990). Advanced mammalian gene transfer: high titre retroviral vectors with multiple drug selection markers and a complementary helper-free packaging cell line. Nucleic Acids Res 18, 3587-3596.
O'Hare, M. J., Bond, J., Clarke, C., Takeuchi, Y., Atherton, A. J., Berry, C., Moody, J., Silver, A. R., Davies, D. C., Alsop, A. E., et al. (2001). Conditional immortalisation of freshly isolated human mammary fibroblasts and endothelial cells. Proc Natl Acad Sci USA 98, 646-651.
Petit, C. A., Gardes, M., and Feunteun, J. (1983). Immortalisation of rodent embryo fibroblasts by SV40 is maintained by the A gene. Virology 127, 74-82.
Rassoulzadegan, M., Perbal, B., and Cuzin, F. (1978). Growth control in simian virus 40-transformed rat cells: temperature-independent expression of the transformed phenotype in tsA transformants derived by agar selection. J Virol 28, 1-5.
Ray, F. A., Peabody, D. S., Cooper, J. L., Cram, L. S., and Kraemer, P. M. (1990). SV40 T antigen alone drives karyotype instability that precedes neoplastic transformation of human diploid fibroblasts. J Cell Biochem 42, 13-31.
Roberts, B. T., Farr, K. A., and Hoyt, M. A. (1994). The Saccharomyces cerevisiae checkpoint gene Bub1 encodes a novel protein kinase. Mol Cell Biol 14, 8282-8291.
Ru, H. Y., Chen, R. L., Lu, W. C., and Chen, J. H. (2002). hBub1 defects in leukemia and lymphoma cells. Oncogene 21, 4673-4679.
Seif, R., and Martin, R. G. (1979). Growth state of the cell early after infection with simian virus 40 determines whether the maintenance of transformation will be A-gene dependent or independent. J Virol 31, 350-359.
Sharp-Baker, H., and Chen, R. H. (2001). Spindle checkpoint protein Bub1 is required for kinetochore localization of Mad1, Mad2, Bub3, and CENP-E, independently of its kinase activity. J Cell Biol 153, 1239-1250.
Shay, J. W., Van Der Haegen, B. A., Ying, Y., and Wright, W. E. (1993). The frequency of immortalisation of human fibroblasts and mammary epithelial cells transfected with SV40 large T-antigen. Exp Cell Res 209, 45-52.
Shichiri, M., Yoshinaga, K., Hisatomi, H., Sugihara, K., and Hirata, Y. (2002). Genetic and epigenetic inactivation of mitotic checkpoint genes hBub1 and hBUBR1 and their relationship to survival. Cancer Res 62, 13-17.
Skoufias, D. A., Andreassen, P. R., Lacroix, F. B., Wilson, L., and Margolis, R. L. (2001). Mammalian mad2 and Bub1/bubR1 recognize distinct spindle-attachment and kinetochore-tension checkpoints. Proc Natl Acad Sci USA 98, 4492-4497.
Srinivasan, A., McClellan, A. J., Vartikar, J., Marks, I., Cantalupo, P., Li, Y., Whyte, P., Rundell, K., Brodsky, J. L., and Pipas, J. M. (1997). The amino-terminal transforming region of simian virus 40 large T and small t antigens functions as a J domain. Mol Cell Biol 17, 4761-4773.
Stewart, N., and Bacchetti, S. (1991). Expression of SV40 large T antigen, but not small t antigen, is required for the induction of chromosomal aberrations in transformed human cells. Virology 180, 49-57.
Stubdal, H., Zalvide, J., Campbell, K. S., Schweitzer, C., Roberts, T. M., and DeCaprio, J. A. (1997). Inactivation of pRB-related proteins p130 and p107 mediated by the J domain of simian virus 40 large T antigen. Mol Cell Biol 17, 4979-4990.
Sullivan, C. S., and Pipas, J. M. (2002). T antigens of simian virus 40: molecular chaperones for viral replication and tumorigenesis. Microbiol. Mol Biol Rev 66, 179-202.
Tang, Z., Bharadwaj, R., Li, B., and Yu, H. (2001). Mad2-Independent inhibition of APCCdc20 by the mitotic checkpoint protein BubR1. Dev Cell 1, 227-237.
Taylor, S. S., Ha, E., and McKeon, F. (1998). The human homologue of Bub3 is required for kinetochore localization of Bub1 and a Mad3/Bub1-related protein kinase. J Cell Biol 142, 1-11.
Taylor, S. S., and McKeon, F. (1997). Kinetochore localization of murine Bub1 is required for normal mitotic timing and checkpoint response to spindle damage. Cell 89, 727-735.
Thomas, J. T., and Laimins, L. A. (1998). Human papillomavirus oncoproteins E6 and E7 independently abrogate the mitotic spindle checkpoint. J Virol 72, 1131-1137.
Thompson, D. A., Belinsky, G., Chang, T. H., Jones, D. L., Schlegel, R., and Munger, K. (1997). The human papillomavirus-16 E6 oncoprotein decreases the vigilance of mitotic checkpoints. Oncogene 15, 3025-3035.
Wang, X., Babu, J. R., Harden, J. M., Jablonski, S. A., Gazi, M. H., Lingle, W. L., de Groen, P. C., Yen, T. J., and van Deursen, J. M. (2001). The mitotic checkpoint protein hBUB3 and the mRNA export factor hRAE1 interact with GLE2p-binding sequence (GLEBS)-containing proteins. J Biol Chem 276, 26559-26567.
Warren, C. D., Brady, D. M., Johnston, R. C., Hanna, J. S., Hardwick, K. G., and Spencer, F. A. (2002). Distinct chromosome segregation roles for spindle checkpoint proteins. Mol Biol Cell 13, 3029-3041.
Woods, C., LeFeuvre, C., Stewart, N., and Bacchetti, S. (1994). Induction of genomic instability in SV40 transformed human cells: sufficiency of the N-terminal 147 amino acids of large T antigen and role of pRB and p53. Oncogene 9, 2943-2950.
Zalvide, J., and DeCaprio, J. A. (1995). Role of pRb-related proteins in simian virus 40 large-T-antigen-mediated transformation. Mol Cell Biol 15, 5800-5810.
Zou, H., McGarry, T. J., Bernal, T., and Kirschner, M. W. (1999). Identification of a vertebrate sister-chromatid separation inhibitor involved in transformation and tumorigenesis. Science 285, 418-422.

We claim:

1. A composition of matter comprising:
a) a SV40 T antigen protein that lacks the ability to bind to the Bub1 protein; or
b) a polynucleotide that encodes the SV40 T antigen protein of a) or its complement; or
c) a recombinant mammalian cell comprising a polynucleotide that encodes T antigen, wherein the expressed T antigen is modified to prevent binding between the T antigen and Bub1; or
d) a cell transformed with the polynucleotide of b).

2. The composition of matter according to claim 1, which is the polynucleotide of b), further comprising the catalytic sub-unit of the telomerase complex.

3. The composition of matter according to claim 1, which is the recombinant mammalian cell of c), wherein the recombinant mammalian cell comprises a polynucleotide that encodes T antigen, wherein the expressed T antigen is modified to prevent binding between the T antigen and Bub1.

4. The composition of matter according to claim 3, wherein the recombinant mammalian cell is a human cell.

5. The composition of matter according to claim 3, wherein the recombinant mammalian cell is pluripotent.

6. The composition of matter according to claim 3, wherein the recombinant mammalian cell is selected from the group consisting of a neuroepithelial cell, a mammary luminal cell, and a mammary fibroblast cell.

7. The composition of matter according to claim 3, wherein the polynucleotide encodes the large T antigen.

8. The composition of matter according to claim 3, wherein the expressed T antigen is temperature-sensitive.

9. The composition of matter according to claim 3, wherein the recombinant mammalian cell is a human somatic cell.

10. The composition of matter according to claim 3, wherein the T antigen has one or more of the amino acid residues 89 to 97 from SEQ ID NO:1 deleted or mutated.

11. The composition of matter according to claim 10, wherein the deleted amino acid residue is one or more of the tryptophan residues at position 91, 94, or 95 of SEQ ID NO:1.

12. The composition of matter according to claim 3, wherein the recombinant mammalian cell further comprises the catalytic sub-unit of the telomerase complex.

13. The composition of matter according to claim 12, wherein the sub-unit is a sub-unit of the human telomerase complex.

14. The composition of matter according to claim 1, which is the transformed cell of d).

15. A method for treating a disorder characterized by cell loss or damage, comprising administering an effective amount of a cell to an individual suffering from the disorder, wherein the cell is:
a) transformed with a polynucleotide encoding a SV40 T antigen protein that lacks the ability to bind to the Bub1 protein; or
b) a recombinant mammalian cell comprising a polynucleotide that encodes T antigen, wherein the expressed T antigen is modified to prevent binding between the T antigen and Bub1.

16. The method according to claim 15, wherein the disorder is a cognitive disorder resulting from brain cell loss or damage.

17. The method according to claim 15, wherein the disorder is selected from the group consisting of Alzheimer's disease or Parkinson's disease.

18. The method according to claim 15, wherein the cell further comprises the catalytic sub-unit of the telomerase complex.

19. A method for producing a transformed cell or recombinant mammalian cell, comprising:
a) transforming a cell with a polynucleotide encoding a SV40 T antigen protein that lacks the ability to bind to the Bub1 protein; or
b) introducing a polynucleotide encoding a T antigen into a mammalian cell, wherein the T antigen is modified to prevent binding between the T antigen and Bub1.
